(19) United States
(12) Patent Application Publication
(10) Pub. No.: US 2025/0291780 A1
(43) Pub. Date: Sep. 18, 2025

(54) System and Method for Flowsheet Population

(71) Applicant: Microsoft Technology Licensing, LLC, Redmond, WA (US)

(72) Inventors: Nathan Bodenstab, Eliot, ME (US); Jerome Tremblay, Saguenay (CA); Arlie E. Coles, Rowlett, TX (US); William Ham, Pittsburgh, PA (US)

(21) Appl. No.: 18/980,436

(22) Filed: Dec. 13, 2024

Related U.S. Application Data
(60) Provisional application No. 63/564,356, filed on Mar. 12, 2024.

(51) Int. Cl. G06F 16/22; G06F 16/21; G06F 16/683

(52) U.S. Cl. CPC G06F 16/2237; G06F 16/211; G06F 16/685

(57) ABSTRACT

A method, computer program product, and computing system for flowsheet population. A structured document conforming to a schema and having a plurality of rows, each row comprising a key and a value, is processed. A plurality of key/value variations associated with an instance of information are identified. A plurality of key/value variation vectors are generated by embedding each key/value variation in a vector. The plurality of key/value variation vectors are combined into a combined vector representing the instance of information. A transcript is segmented into a segment, the segment corresponding to the instance of information. A transcript segment vector is generated by embedding the instance of information in the segment into a vector. A similarity between the transcript segment vector and the and the combined vector is determined. The instance is extracted from the transcript segment vector by processing a prompt with a generative artificial intelligence (AI) model using retrieval augmented generation (RAG). A value of a row corresponding to the key is populated with the instance of information.

### RELATED CASES

This application claims the benefit of U.S. Provisional Application No. 63/564,356 filed on Mar. 12, 2024, the contents of which are all incorporated by reference.

### BACKGROUND

In the context of documentation application for patient care, nurses must complete a flowsheet in the electronic health record (EHR) to document and track a patient's clinical data over time. This flowsheet contains many rows that can be filled with different kinds and types of information about the patient (e.g. blood pressure, temperature, etc.). All rows that can be filled with information are defined in a schema that is specific to each hospital. This hospital-specific schema can contain thousands of rows, but in a specific context only a small number of rows are relevant. Manually determining which rows are relevant and inserting appropriate data in those rows is reduces the efficacy of nursing personnel.

### BRIEF DESCRIPTION OF THE DRAWINGS

FIG. 1 is a diagrammatic view of a flowsheet population system;

FIG. 2 is a depicts a sample structured document (e.g., a flowsheet);

FIG. 3 is a flowchart of an embodiment of the flowsheet population process;

FIG. 4 depicts forms information in a transcript and in an associated structured flowsheet;

FIG. 5 is a flowchart of an embodiment of the flowsheet population process;

FIG. 6 is a block diagram of an embodiment of a multiple vector embedding process; and

FIG. 7 is a diagrammatic view of a computer system and the operating system and flowsheet population process coupled to a distributed computing network.

Like reference symbols in the various drawings indicate like elements.

### DETAILED DESCRIPTION

Documentation in many fields is defined by a structured document or flowsheet. In the example field of healthcare, medical or clinical documentation is tracked in an electronic medical record (EHR), where a user or healthcare professional updates the education provided, updates the care team with progress notes regarding the patient's condition, documents significant events, and details medical assessments in structured documents.

A flowsheet is a structured document used in various fields, including healthcare, engineering, and manufacturing, to record and track the progression of processes, procedures, and/or events over time. In healthcare, a flowsheet is commonly used to document and monitor patient data, particularly vital signs, measurements, and clinical assessments, in a systematic and organized manner. It typically consists of a grid or table format with rows representing different parameters or variables being measured and columns representing different time intervals, such as hours, days, or work shifts.

Each flowsheet can have multiple tabs and hundreds or even thousands of rows, with each row representing a specific data point. Working with flowsheets is a time-consuming process, since the user must first navigate to the appropriate tab in the flowsheet, identify the row or group of rows corresponding to the assessment that is being documented, then enter the information cell-by-cell. Health care professionals often do not document in flowsheets while delivering care and instead postpone this task, which can increase cognitive burden, extend the time it takes to chart, delay when this information is accessible to other clinicians, and increase the potential for inaccuracies. Within a healthcare setting, flowsheets may include categories for recording vital signs such as blood pressure, heart rate, temperature, respiratory rate, oxygen saturation, etc. Additionally, flowsheets may incorporate clinical assessments, interventions, medications administered, fluid intake and output, and other relevant patient information.

### Flowsheet Population

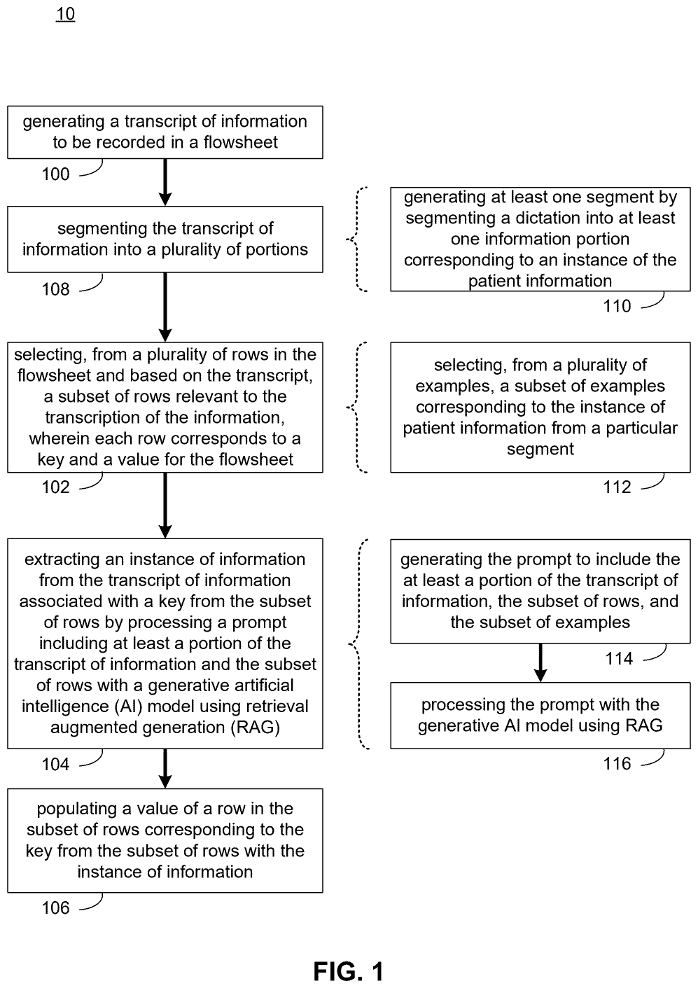

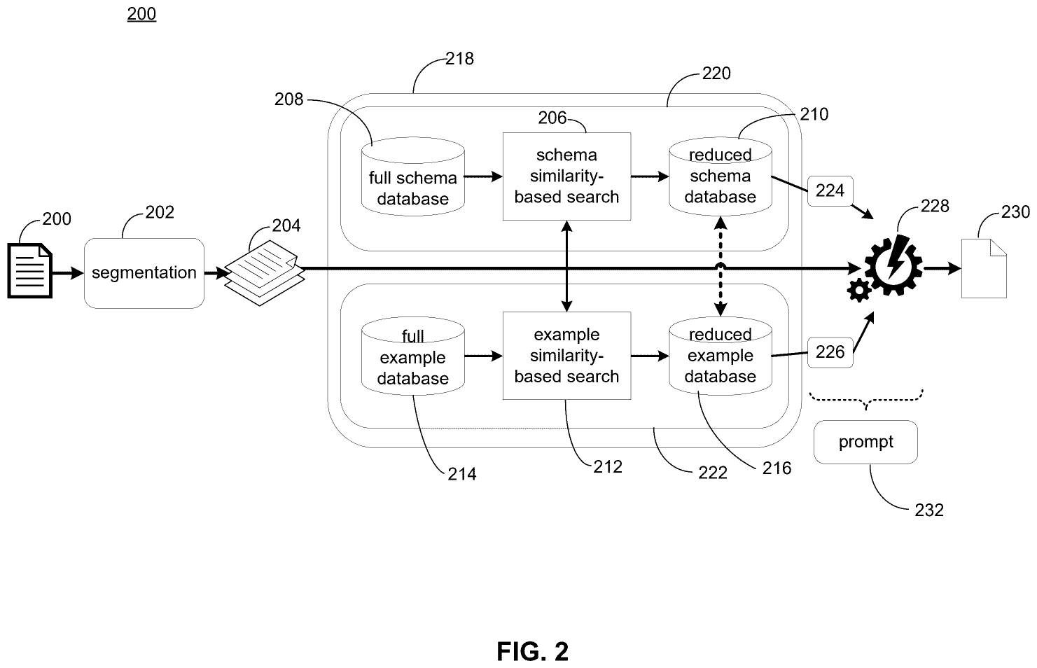

Referring to FIG. 1, a flowchart describing an implementation of flowsheet population process 10 is shown. In some implementations, flowsheet population process 10 generates 100 a transcript of information that includes instances of information that are to be inserted into a flowsheet, such as flowsheet 200 of FIG. 2. Flowsheet population process 10 selects 102, from a plurality of rows in the flowsheet and based on the transcript of information, a subset of rows relevant to the transcript of the information, wherein each row corresponds to a key and a value for the flowsheet. An instance of information is extracted 104 from the transcript of information associated with a key from the subset of rows by processing a prompt including at least a portion of the transcript of information and the subset of rows with a generative artificial intelligence (AI) model using retrieval augmented generation (RAG). A value of a row in the subset of rows corresponding to the key from the subset of rows is populated 106 with the instance of information.

Referring again to FIG. 1, flowsheet population process 10 generates 100 a transcript of information (e.g., transcript 200) through processing, transcription, and diarization of speech signals from one or more users. In some implementations, an optimized Speech-to-Text artificial intelligence (AI) model converts natural input speech from one, or multiple speakers, to textual form. In one example, the transcript is generated using an automated speech recognition (ASR) system from a dictation from a healthcare provider. In another example, the transcript is generated using an ASR system from a conversation between at least one healthcare provider and a patient. The input speech sequence may contain both meaningful and non-meaningful parts for flowsheet documentation. Meaningful speech includes verbalizations which are directly relevant to the flowsheet fields for a given patient, while non-meaningful speech includes any other irrelevant speech to the flowsheet documentation. The information extraction step that follows speech-to-text, can extract the meaningful information for the flowsheet documentation and reject the irrelevant information.

The audio capture device can be a mobile phone, in-room microphone array, desktop microphone, wearable device, or any other audio capture device. In some implementations, a recording device is positioned adjacent to the two speakers to capture the audio from both parties, an omnidirectional microphone can capture all audio in the vicinity, and an artificial intelligence (AI) model can diarize the speech by both speakers and ASR can transcribe all speech.

A microphone array may be used, in which the room can be equipped with wall mount microphone array. Alternatively, a portable microphone array built-in to a computing device is used. In one example, the microphone array is used to track non-stationary speakers by beamforming technology. In such a scenario, target speakers are pre-enrolled by voice biometrics. The diarization process tags target speakers, whose voice biometrics match, and other all non-enrolled speakers' speech are marked as unrecognized speakers. In one example, the ASR system transcribes only targeted speakers' speech.

In some implementation, flowsheet population process 10 segments 108 a first instance of information corresponding to a key and an associate value. For example and as discussed above, flowsheet population process 10 segments 108 the streaming or recorded transcript into contextually coherent, continuous and possibly overlapping segments such as phenomes, words, or phrases, via an artificial intelligence (AI) model. In one example, a prompted large language model (LLM) is instructed to divide the input text into contiguous segments, with the goal of keeping all related information together in one segment. In some implementations, segmenting 108 includes identifying pauses, changes in pitch, or other acoustic features that indicate the boundaries between different linguistic units. Effective segmentation improves the accuracy of speech recognition by ensuring that the system can better handle variations in speech, such as different speaking speeds, accents, and sentence structures. It also aids in improving the alignment between spoken words and their corresponding text, facilitating more precise transcriptions in ASR systems.

In some implementations, segmenting 108 the first instance of information includes generating 110 at least one segment by segmenting the dictation into at least one information portion corresponding to an instance of the patient information. As discussed above and in the example where the flowsheet concerns patient information for a healthcare provider, flowsheet population process 10 processes a dictation prepared by or for a healthcare professional concerning a patient. In this example, flowsheet population process 10 generates 110 segments the dictation into information portions or segments corresponding to particular instances of patient information. Referring also to FIG. 2 and in some implementations, the transcript is broken down into segments by a segmentation process 202, which decomposes the transcript into smaller substrings prior to processing.

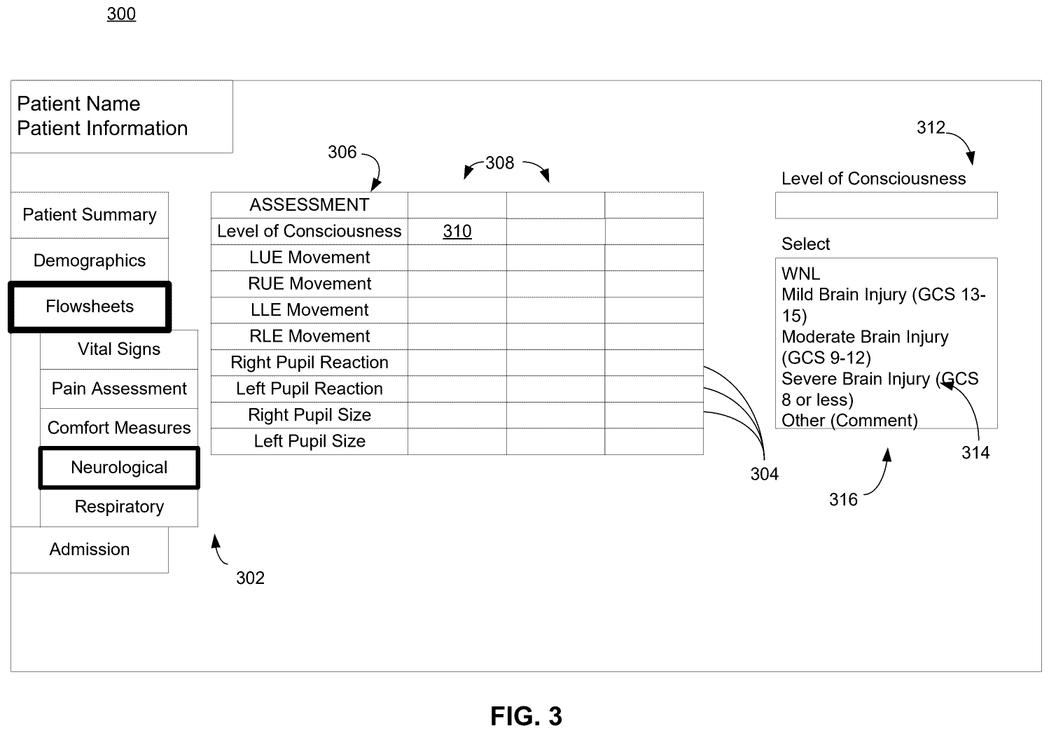

In some implementations, flowsheet population process 10 selects 102, from a plurality of rows in the flowsheet and based on the transcript, a subset of rows relevant to the transcript of the information. Referring also to FIG. 3, an example flowsheet 300 according to an embodiment of the disclosure is shown. Flowsheet 300 includes a listing of groups of data 302 associated with various information that will be contained in the flowsheet. Within each group, a number of rows 304 include key/value information for specific instances of information, designated at column 306. Within each column 308, information for different instances during an observation period are input for each row. Field 310 shows an example field into which a value would be input corresponding to the key “Level of consciousness.” Information may be input into field 310 (or any other field in the flowsheet) in the form of an integer, a float figure, string, date, time, selection from a single-choice list, a selection from a multi-choice list, or any other custom data type. In flowsheet 300, section 312 indicates a field in which a variable or freeform input may be entered. Window 314 is a multi-choice list, from which a user may select the input, and field 316 allows for the entry of comments in addition to the value input. In some implementations, flowsheet population process 10 uses a schema similarity-based search (e.g., schema similarity-based search section 206), to identify the most relevant rows in a full schema database (e.g., full schema database 208) and stores them in the reduced schema database 210 for use in connection with matching information in each segment with the appropriate row (key/value pair) in the flowsheet.

In some implementations, selecting 102 the subset of rows includes selecting 112, from a plurality of examples, a subset of examples corresponding to the instance of patient information from a particular segment. For instance and as shown in FIG. 2, flowsheet population process 10 performs example similarity-based search (e.g., example similarity-based search section 212) using a database (e.g., database 214) of examples for storing the full set of examples. In one example, flowsheet population process 10 processes segments of an input transcript to identify the most relevant examples in the full example database 214 and stores them in the reduced example database 216 for use in connection with matching information in each segment with the appropriate row (key/value pair) in the flowsheet corresponding to instances of patient information.

For example and referring again to FIG. 2, flowsheet population process 10 selects 102 the subset of rows using a retrieval augmented generation (RAG) portion 218, including a schema row selection section 220 and an in-context example selection section 222. Schema row selection section 220 includes a database 208 for storing the full schema of the hospital flowsheet, which can include thousands of rows of the flowsheet. A schema similarity-based search section 206 processes segments of an input transcript to identify most relevant rows in the full schema database 208 and stores them in the reduced schema database 210 for use in connection with matching information in each segment with the appropriate row (key/value pair) in the flowsheet. Example selection section 222 includes a database 214 for storing the full set of examples. An example similarity-based search section 212 processes segments of an input transcript to identify the most relevant examples in the full example database 214 and stores them in the reduced example database 216 for use in connection with matching information in each segment with the appropriate row (key/value pair) in the flowsheet.

In some implementations, the transcript segments 204 are input to schema similarity-based search section 206 and example similarity-based search section 212 for use in determining into which rows 224 the instances of information will be inserted and to determine which examples 226 would be beneficial in making the row-similarity decisions. Similarity-based search is used to retrieve items or data in each database 208, 214 that are similar to each input segment, based on specific features or characteristics of the segment. Rather than searching for exact matches, this approach uses algorithms to measure the degree of similarity between the input segment and the items in the databases 208, 214. The goal is to find rows and examples from databases 208 and 214, respectively, that share certain patterns, structures, or features with the input segment. For instance, in image search, a similarity-based search might compare visual features like color, shape, or texture to find images that resemble a target image. This type of search is valuable when exact matches are rare or insufficient, allowing for more flexible and intelligent retrieval of relevant results.

In some implementations and in the example of FIG. 2, rows 224 represent a subset of the rows in the full schema database 208. Based on the similarity-based search 206, the rows in the flowsheet having key/value pairs most similar to the segment being processed are selected and stored in reduced schema database 210. Likewise, based on the similarity-based search 212, the examples in full example database 214 that are most similar to the segment being processed are selected and stored in reduced examples database 216.

In some implementations, flowsheet population process 10 extracts 104 an instance of information from the transcript of information associated with a key from the subset of rows by processing a prompt including at least a portion of the transcript of information and the subset of rows with a generative artificial intelligence (AI) model using retrieval augmented generation (RAG). RAG combines the strengths of retrieval-based methods and generative models to enhance the quality and relevance of generated text. In RAG, a generative AI model, typically a large language model, is augmented with a retrieval component, allowing it to retrieve and incorporate relevant information from a large external knowledge source, such as full schema database 208, during the generation process. The retrieval component retrieves relevant context based on the input prompt or query, which is then used to guide the generation of text by the generative model. By leveraging external knowledge, RAG models can produce more informative, coherent, and contextually relevant responses compared to traditional generative models. Moreover, the retrieval mechanism enables RAG models to handle open-domain conversational tasks more effectively, as they can access a broader range of information beyond what is explicitly encoded in their parameters.

In some implementations, determined rows 224, determined examples 226, and segments 204 are input into a prompt of a large language model (e.g., generative AI model 228), which performs an extraction process to extract data from the segments and, as will be discussed in greater detail below, generate the flowsheet with the instances of information from each segment populated into appropriate rows of the flowsheet. In one example, the flowsheet is a structured JSON document 230.

To reduce latency and cost, and to stay within downstream context window constraints, the size of the schema to extract into is minimized by filtering potential rows to the top, for example, “N” candidates given the current segment with a retrieval-augmented generation (RAG) filtering method 220. The top “N” candidate rows are generated by measuring the similarity (e.g., cosine similarity, Euclidean distance, dot product similarity, etc.) between the segment 204 and each schema row, projecting each into generative AI model 228's embedding space. For example, the “N” most similar rows identified in schema similarity-based search process 110 are used to create a just-in-time prompt (e.g., prompt 234) for later data extraction, containing only relevant schema components.

In some implementations, extracting 104 the instance of information includes: generating 114 the prompt to include the at least a portion of the transcript of information, the subset of rows, and the subset of examples; and processing 116 the prompt with the generative AI model using RAG. For example, prompt 234 is generated to include in-context examples 226. An in-context example refers to an input-output pair (e.g. an input spoken text segment and the corresponding output structured data). These examples are useful for improving the robustness and generalization capabilities of structured data extraction, where variability in speaking patterns may change from user to user or over time.

In some implementations, when a new hospital is being adapted for processing according to embodiments of the disclosure, the hospital's schema is provided but no supervised examples may exist to retrieve for in-context learning. In this situation, a generative AI model (e.g., generative AI model 228) is leveraged to generate synthetic examples for each row in the hospital schema with the help of an existing example database for a sample schema. In some implementations, this is done in two steps. First, for each row in the hospital schema, a row embedding is generated, then its most similar rows from the sample schema are selected by the similarity (e.g., cosine similarity, Euclidean distance, dot product similarity, etc.) of their embeddings. Second, generative AI model 228 is prompted with examples containing those rows from the sample schema, using, for example, chain-of-thought prompting to force the model to generate a mapping, step-by-step, by first identifying the specific attributes of the row (e.g. datatype, value) that it must use before generating an appropriate transcript to be paired with the corresponding similar hospital schema row.

A generative AI model 228 (e.g., a prompted large language model (LLM)) is used to extract 104 the relevant structured text from the transcript segment 204, rows 224, and examples 226, and canonicalize it based on the schema. The extraction process isolates the needed information while discarding irrelevant data. Canonicalization follows extraction and involves standardizing the data into a consistent and uniform format, regardless of its original structure. In one example, this includes normalizing variations in data representation, such as converting all dates into a single format, standardizing case sensitivity, or resolving different identifiers for the same entity (e.g., “NYC” and “New York City”). Canonicalization ensures that all extracted data conforms to the flowsheet schema, despite how it was spoken. For example, the segment “the patient has a risk of fracture in his left arm” is converted to {“BoneFractureRiskOrInjury”: “1=Yes”} as the schema specifies two pick-list items for this row as the strings “0=No” and “1=Yes”.

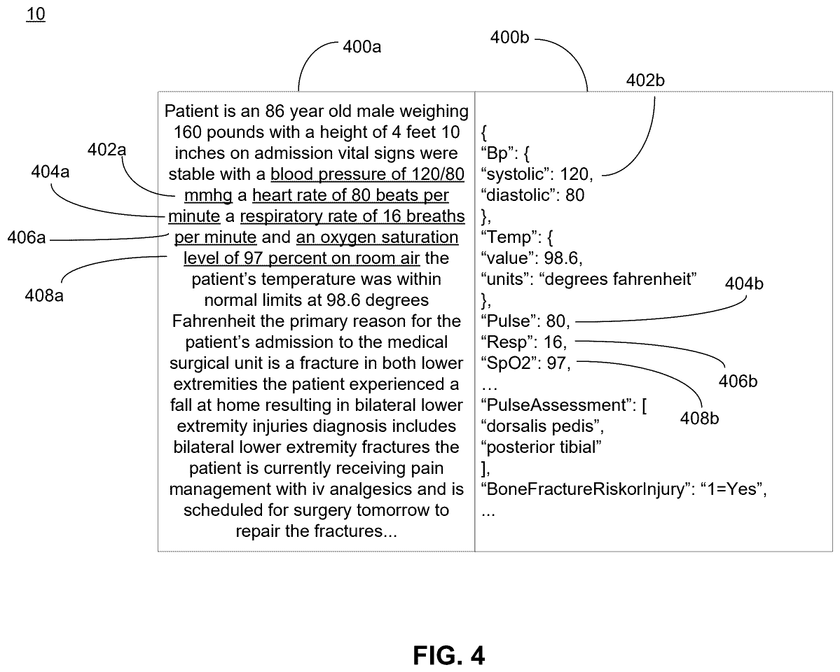

In some implementations, flowsheet population process 10 populates 106 a row in the subset of rows corresponding to the key of the first instance of information with the associated value. For example, FIG. 4 depicts an example of flowsheet population process 10, in which instances of information in a transcript are populated into rows (key/value) of a structured document (e.g., flowsheet). A portion 400a of a transcript is shown, along with the corresponding portion 400b of the structured flowsheet. Indicated at 402a is a segment of the transcript 400a indicating a “blood pressure of 120/80 mmhg.” After processing of the transcript 400a as described above, the information in this segment 402a is inserted into the key and value fields in the associated row 402b of the flowsheet 400b. In the associated row 402b in the flowsheet 400b, the key “Bp” (for blood pressure) is inserted and the values 120 (systolic) and 80 (diastolic) are entered. Likewise, the instance of information in segment 404a is inserted into the key and value fields in the associated row 404b of the flowsheet 400b. In the associated row 404b in the flowsheet 400b, the key “Pulse” and the value “80” are entered. The instance of information in segment 406a is inserted into the key and value fields in the associated row 406b of the flowsheet 400b. In the associated row 406b in the flowsheet 400b, the key “Resp” and the value “16” are entered. In this example, the instance of information in segment 408a is inserted into the key and value fields in the associated row 408b of the flowsheet 400b. In the associated row 408b in the flowsheet 400b, the key “SpO2” and the value “97” are entered. In this manner, flowsheet population process 10 automatically populates rows of the flowsheet by mapping segments of a transcript to key/value portions of the schema of the flowsheet.

While embodiments of the disclosure have been described mainly with regard to the application to hospital flowsheets for use by medical personnel, the system and method described herein may be utilized in connection with any environment in which freeform speech or unstructured text information is to be entered into a structured document conforming to a schema.

### Embedding Multiple Vectors

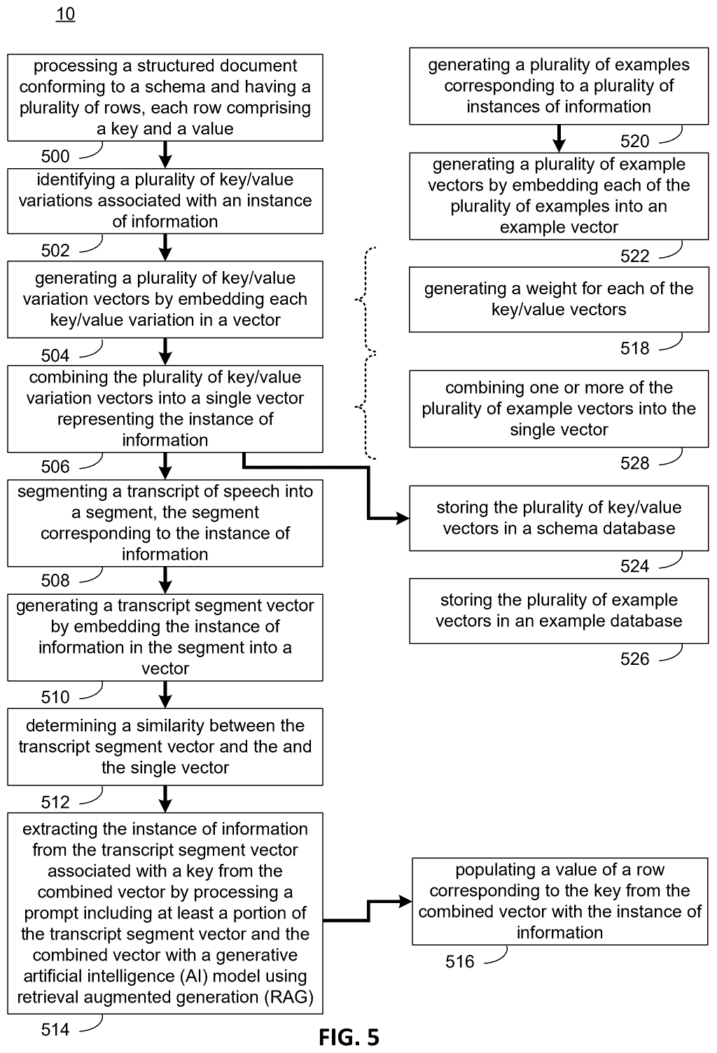

Referring also to FIG. 5, a flowchart describing an implementation of flowsheet population process 10 is shown. In some implementations, flowsheet population process 10 processes 500 a structured document conforming to a schema and having a plurality of rows, each row comprising a key and a value. A plurality of key/value variations associated with an instance of information are identified 502. A plurality of key/value variation vectors are generated 504 by embedding each key/value variation in a vector. The plurality of key/value variation vectors are combined 506 into a combined vector representing the instance of information. A transcript of speech is segmented 508 into a segment, the segment corresponding to the instance of information. A transcript segment vector is generated 510 by embedding the instance of information in the segment into a vector. A similarity between the transcript segment vector and the and the combined vector is determined 512. The instance of information is extracted 514 from the transcript segment vector associated with a key from the combined vector by processing a prompt including at least a portion of the transcript segment vector and the combined vector with a generative artificial intelligence (AI) model using retrieval augmented generation (RAG). A value of a row corresponding to the key from the combined vector is populated 516 with the instance of information.

As discussed above, flowsheet population process 10 selects 102, from a plurality of rows in the flowsheet and based on the transcript, a subset of rows relevant to the transcript of the information. To improve the embedding quality, flowsheet population process 10 selects 112, from a plurality of examples, a subset of examples corresponding to the instance of patient information from a particular segment. However, instead of embedding example information in a straightforward way (i.e., a single string embedding of all the information), flowsheet population process 10 generates an embedding from each component and combines these embeddings in a weighted average approach.

In some implementations, flowsheet population process 10 processes 500 a structured document conforming to a schema and having a plurality of rows, each row comprising a key and a value. For example and as described above, a structured document conforms to a schema with various rows. In one example, the structured document is a flowsheet with rows that correspond to patient information. With each row of the schema, there is a key and a value. The key is a unique identifier for a record in a database and the value is the actual data stored in the record associated with the key.

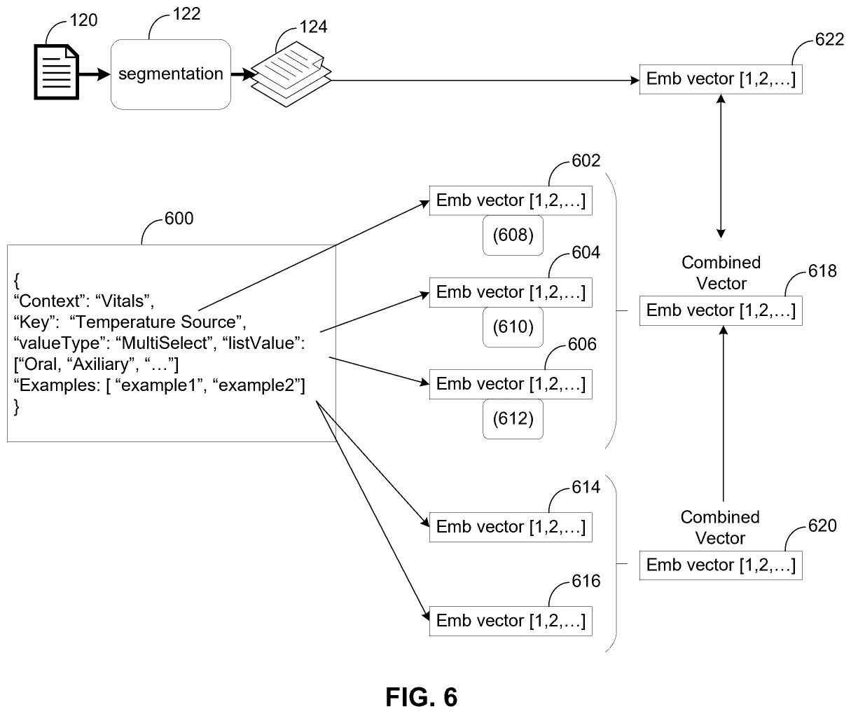

In some implementations, flowsheet population process 10 identifies 502 a plurality of key/value variations associated with an instance of information. Referring also to FIG. 6 and in some implementations, flowsheet population process 10 identifies 502 key/value variations associated with a particular instance of information (i.e., temperature in this example). In the example of FIG. 6, the plurality of key/value variations are identified 502 from a structured document (e.g., structured document 600). While FIG. 6 shows a single example, it will be appreciated that many combined vectors are possible within the scope of the present disclosure. For example, implementations of the present disclosure likely have thousands of possible combined vectors and the closest “N” are found using a schema similarity-based search using a segment vector, as described below.

In some implementations, flowsheet population process 10 generates 504 a plurality of key/value variation vectors by embedding each key/value variation in a vector. Generating 504 key/value variation vectors includes converting each key/value variation into a numerical representation of the semantic properties of the key/value variation. For instance, flowsheet population process 10 generates 504 key/value variation vectors 602, 604, 606, 608 for key/variations from structured document 600 by using a natural language processing (NLP) engine to generate semantic representations of each key/value variation.

In some implementations, generating 504 the plurality of key/value variation vectors includes generates 518 a weight for each of the key/value variation vectors. For example, weights can be set manually (e.g., by a user) or derived by a generative AI model from training data to weight each component (i.e., key/value variation) in the composition (i.e., in the plurality of key/value variations). In this example, flowsheet population process 10 generates 518 a weight for each key/value variation vector (e.g., weight 608 for key/value variation vector 602; weight 610 for key/value variation vector 604; and weight 612 for key/value variation vector 606).

In some implementations, flowsheet generation process 10 generates 520 a plurality of examples corresponding to a plurality of instances of information. For example and as discussed above, flowsheet population process 10 performs example similarity-based search (e.g., example similarity-based search section 116) using a database (e.g., database 114) of examples for storing a set of examples. In some implementations, each example includes a text input corresponding to an instance of information. For instance, these examples are generated for the purpose of enabling variations in the form of an instance of information input to the transcript to account for (e.g., different healthcare personnel reporting the instance of information in a strict manner as opposed to a casual manner, or any other difference in how the instances of information are entered). Referring again to the example of FIG. 6, flowsheet population process 10 may generate 520 examples from the examples in structured document 600 (e.g., “example1”; example2”, etc.). In some implementations, flowsheet population process 10 generates 522 a plurality of example vectors by embedding each of the plurality of examples into an example vector. For example and as discussed above concerning key/value variation vectors, flowsheet population process 10 generates 522 a plurality of example vectors (e.g., example vectors 614, 616) by converting each example into a numerical representation of the semantic properties of the example.

In some implementations, flowsheet population process 10 stores 524 the plurality of key/value vectors in a schema database. For example and as discussed above, flowsheet population process 10 maintains a full schema database (e.g., full schema database 108). In some implementations, flowsheet population process 10 stores 524 the plurality of key/value vectors in full schema database 108. Similarly and in some implementations, flowsheet population process 10 stores 526 the plurality of example vectors in an example database. For example and as discussed above, flowsheet population process 10 maintains a full example database (e.g., full example database 114). In some implementations, flowsheet population process 10 stores 526 the plurality of example vectors in full example database 114.

In some implementations, flowsheet population process 10 combines 506 the plurality of key/value variation vectors into a combined vector representing the instance of information. For example, flowsheet population process 10 combines 506 each vector by multiplying the value of each position in the vector by its weight defined for the key/value variation vector. The respective values of each key/value variation vector are combined 506 by averaging the values across each key/value variation vector into a single value. In some implementations, this is a weighted average using the weights of each key/value variation vector. Referring again to FIG. 6, flowsheet population process 10 combines 506 key/value variation vectors 602, 604, 606 into a combined vector (e.g., combined vector 618). In some implementations, flowsheet population process 10 combines 506 key/value variation vectors 602, 604, 606 into combined vector 618 using weights 608, 610, 612.

In some implementations, combining 506 the plurality of key/value variation vectors into the combined vector includes combining 528 one or more of the plurality of example vectors into the combined vector. For example and in a similar manner to combining key/value variation vectors, flowsheet population process 10 combines 528 example vectors 614, 616 into combined example vector 620. In some implementations, flowsheet population process 10 adds combined example vector 620 to combined vector 618. In other words, example vectors that correspond to particular instances of information are added into combined vectors that represent a plurality of key/value variation vectors corresponding to the particular instance of information.

In some implementations, flowsheet population process 10 segments 508 a transcript of speech into a segment, the segment corresponding to the instance of information. For example and as discussed above, flowsheet population process 10 segments 508 (e.g., as shown in FIG. 1 as segmentation step 122) the transcript into contextually coherent, continuous and non-overlapping segments such as phenomes, words, or phrases, via a generative artificial intelligence (AI) model. In some implementations, segmenting 508 includes identifying pauses, changes in pitch, or other acoustic features that indicate the boundaries between different linguistic units. Effective segmentation improves the accuracy of speech recognition by ensuring that the system can better handle variations in speech, such as different speaking speeds, accents, and sentence structures. It also aids in improving the alignment between spoken words and their corresponding text, facilitating more precise transcriptions in ASR systems. To reduce variables, segmentation is kept constant for all implementations, although this is not required.

In some implementations, segmenting 508 the transcript includes generating at least one segment by segmenting the dictation into at least one information portion corresponding to an instance of the patient information. As discussed above and in the example where the flowsheet concerns patient information for a healthcare provider, flowsheet population process 10 processes a dictation prepared by or for a healthcare professional concerning a patient. In this example, flowsheet population process 10 generates segments the dictation into information portions corresponding to particular instances of patient information.

In some implementations, flowsheet population process 10 generates 510 a transcript segment vector by embedding the instance of information in the segment into a vector. For example, when processing a transcript, flowsheet population process 10 generates 510 a vector for each transcript segment by generating an embedding of the instance of information from the transcript segment into a numerical representation. In the example of FIG. 6, flowsheet population process 10 generates transcript segment vector 622 from segments 124. In this manner, flowsheet population process 10 generates 510 vector representation of each segment of the transcript for comparing against the combined vector for similarity.

In some implementations, flowsheet population process 10 determines 512 a similarity between the transcript segment vector and the combined vector. In one example, flowsheet population process 10 determines 512 the similarity between transcript segment vector 622 and combined vector 618 by determining a Euclidean distance between the transcript segment vector 622 and combined vector 618 as the straight-line distance between two vectors in a multidimensional space. In another example, flowsheet population process 10 determines 512 the similarity between transcript segment vector 622 and combined vector 618 by determining a dot product similarity between the transcript segment vector 622 and combined vector 618, where the dot product similarity is determined by adding the products of the vectors' corresponding components. In another example, flowsheet population process 10 determines 512 the similarity between transcript segment vector 622 and combined vector 618 by determining a Cosine similarity between the transcript segment vector 622 and combined vector 618, where the cosine similarity is a measure of the angle between two vectors. In some implementations, flowsheet population process 10 determines that the transcript segment vector and the combined vector based upon, at least in part, a predefined threshold. In this manner, flowsheet population process 10 identifies key/value variations that correspond to the instance of information embedded in the transcript segment vector.

In some implementations, flowsheet population process 10 extracts 514 the instance of information from the transcript segment vector associated with a key from the combined vector by processing a prompt including at least a portion of the transcript segment vector and the combined vector with generative AI model 228 using RAG. As described above, flowsheet population process 10 generates a prompt to include transcript segment vector 622 and combined vector (e.g., embedding vector 618 and embedding vector 620) and processes the prompt on generative AI model 228 using RAG to extract an instance of information from transcript segment vector 622 associated with a key from the combined vector.

In some implementations, flowsheet population process 10 populates 516 a value of a row corresponding to the key from the combined vector with the instance of information. For example and in response to determining that the transcript segment vector (e.g., transcript segment vector 622) and the combined vector (e.g., combined vector 618) are sufficiently similar, flowsheet population process 10 populates a value from the key/value from the combine vector with the instance of information embedded in the transcript segment vector. In one example, flowsheet population process 10 embeds a schema-like structured input by embedding a string representation of “Key=Arm/Leg Bands On, Value=SingleSelect [Yes|No] and extracts the individual components from the structured data for encoding (e.g., “Arm/Leg Bands On Yes No”). In some implementations, flowsheet population process 10 computes a single embedding composed of multiple instantiation variations, by creating each of these variations, embedding each in a vector (e.g., an “m”-dimensional vector), then composing all m-dimensional vectors into a combined vector representing the schema entry.

Continuing with this example, suppose that there are four key/value variations: Arm/Leg Bands On Yes; Arm/Leg Bands On No; Yes Arm Bands On; No Leg Bands On. In this example, flowsheet population process 10 generates weights for each key/value variation and combines the plurality of key/value variation vectors into a combined vector.

In some implementations, when a full schema database and a full example database are available, flowsheet population process 10 accesses existing pairs of input text mapped to extraction results and uses these mapped pairs to generate additional schema embeddings. In this example, the input text is used as another, weighted input to a combined vector (e.g., with additional variations for the text “the leg bands were applied”; and “both arm and leg straps were securely fastened”).

While embodiments of the disclosure have been described mainly with regard to the application to hospital flowsheets for use by medical personnel, the system and method described herein may be utilized in connection with any environment in which freeform speech information is to be entered into a structured document.

### System Overview:

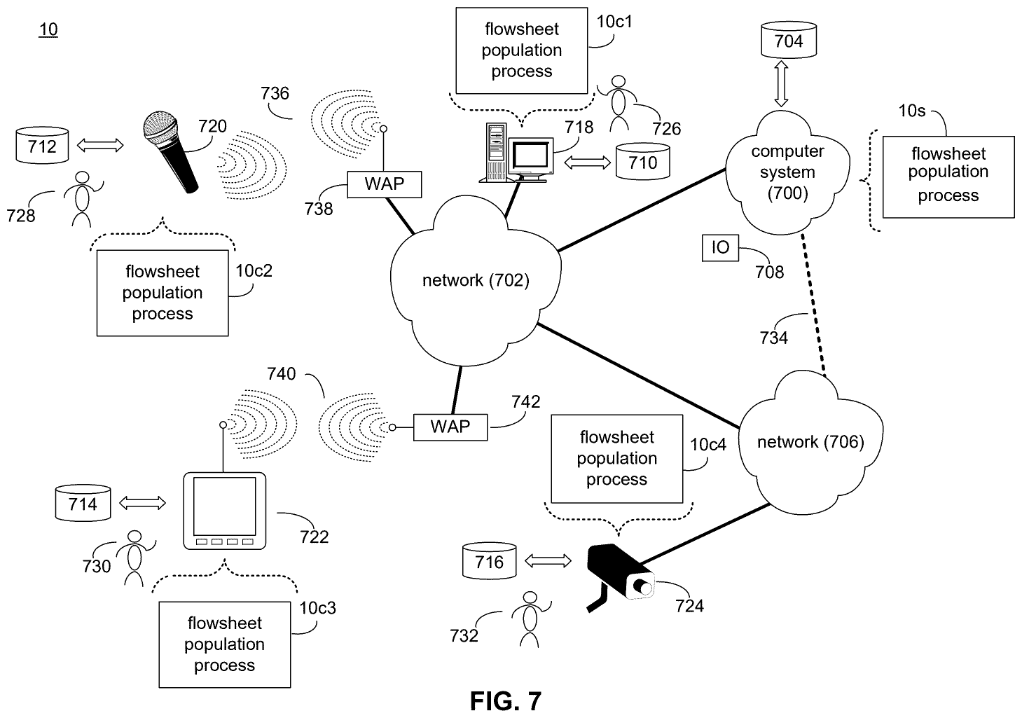

Referring to FIG. 7, there is shown a flowsheet population process 10. Flowsheet population process 10 may be implemented as a server-side process, a client-side process, or a hybrid server-side/client-side process. For example, flowsheet population process 10 may be implemented as a purely server-side process via flowsheet population process 10s. Alternatively, flowsheet population process 10 may be implemented as a purely client-side process via one or more of flowsheet population process 10cl, flowsheet population process 10c2, flowsheet population process 10c3, and flowsheet population process 10c4. Alternatively still, flowsheet population process 1044 may be implemented as a hybrid server-side/client-side process via flowsheet population process 10s in combination with one or more of flowsheet population process 10cl, flowsheet population process 10c2, flowsheet population process 10c3, and flowsheet population process 10c4.

Accordingly, flowsheet population process 10 as used in this disclosure may include any combination of flowsheet population process 10, flowsheet population process 10cl, schema matching process, flowsheet population process 10c3, and flowsheet population process 10c4.

Flowsheet population process 10s may be a server application and may reside on and may be executed by a computer system 700, which may be connected to network 702 (e.g., the Internet or a local area network). Computer system 700 may include various components, examples of which may include but are not limited to: a personal computer, a server computer, a series of server computers, a mini computer, a mainframe computer, one or more Network Attached Storage (NAS) systems, one or more Storage Area Network (SAN) systems, one or more Platform as a Service (PaaS) systems, one or more Infrastructure as a Service (IaaS) systems, one or more Software as a Service (SaaS) systems, a cloud-based computational system, and a cloud-based storage platform.

A SAN includes one or more of a personal computer, a server computer, a series of server computers, a minicomputer, a mainframe computer, a RAID device and a NAS system. The various components of computer system 700 may execute one or more operating systems.

The instruction sets and subroutines of computational cost reduction process 10s, which may be stored on storage device 704 coupled to computer system 700, may be executed by one or more processors (not shown) and one or more memory architectures (not shown) included within computer system 700. Examples of storage device 704 may include but are not limited to: a hard disk drive; a RAID device; a random-access memory (RAM); a read-only memory (ROM); and all forms of flash memory storage devices.

Network 702 may be connected to one or more secondary networks (e.g., network 704), examples of which may include but are not limited to: a local area network; a wide area network; or an intranet, for example.

Various IO requests (e.g., IO request 708) may be sent from flowsheet population process 10s, flowsheet population process 10c1, flowsheet population process 10c2, flowsheet population process 10c3 and/or flowsheet population process 10c4 to computer system 700. Examples of IO request 708 may include but are not limited to data write requests (i.e., a request that content be written to computer system 700) and data read requests (i.e., a request that content be read from computer system 700).

The instruction sets and subroutines of flowsheet population process 10cl, flowsheet population process 10c2, flowsheet population process 10c3 and/or flowsheet population process 10c4, which may be stored on storage devices 710, 712, 714, 716 (respectively) coupled to client electronic devices 718, 720, 722, 724 (respectively), may be executed by one or more processors (not shown) and one or more memory architectures (not shown) incorporated into client electronic devices 718, 720, 722, 724 (respectively). Storage devices 710, 712, 714, 716 may include but are not limited to: hard disk drives; optical drives; RAID devices; random access memories (RAM); read-only memories (ROM), and all forms of flash memory storage devices. Examples of client electronic devices 718, 720, 722, 724 may include, but are not limited to, personal computing device 718 (e.g., a smart phone, a personal digital assistant, a laptop computer, a notebook computer, and a desktop computer), audio input device 720 (e.g., a handheld microphone, a lapel microphone, an embedded microphone (such as those embedded within eyeglasses, smart phones, tablet computers and/or watches) and an audio recording device), display device 722 (e.g., a tablet computer, a computer monitor, and a smart television), a hybrid device (e.g., a single device that includes the functionality of one or more of the above-references devices; not shown), an audio rendering device (e.g., a speaker system, a headphone system, or an earbud system; not shown), and a dedicated network device (not shown).

Users 726, 728, 730, 732 may access computer system 700 directly through network 702 or through secondary network 706. Further, computer system 700 may be connected to network 702 through secondary network 706, as illustrated with link line 734.

The various client electronic devices (e.g., client electronic devices 718, 720, 722, 724) may be directly or indirectly coupled to network 702 (or network 706). For example, personal computing device 718 is shown directly coupled to network 702 via a hardwired network connection. Further, machine vision input device 724 is shown directly coupled to network 706 via a hardwired network connection. Audio input device 722 is shown wirelessly coupled to network 702 via wireless communication channel 736 established between audio input device 720 and wireless access point (i.e., WAP) 738, which is shown directly coupled to network 702. WAP 738 may be, for example, an IEEE 802.11a, 802.11b, 802.11g, 802.11n, Wi-Fi, and/or any device that is capable of establishing wireless communication channel 736 between audio input device 720 and WAP 738. Display device 722 is shown wirelessly coupled to network 702 via wireless communication channel 740 established between display device 722 and WAP 742, which is shown directly coupled to network 702.

The various client electronic devices (e.g., client electronic devices 718, 720, 722, 724) may each execute an operating system, wherein the combination of the various client electronic devices (e.g., client electronic devices 718, 720, 722, 724) and computer system 700 may form a modular system.

### General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be used. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet.

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, not at all, or in any combination with any other flowcharts depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. As used herein, the language “at least one of A and B” (and the like) as well as “at least one of A or B” (and the like) should be interpreted as covering only A, only B, or both A and B, unless the context clearly indicates otherwise. The language “one or more of A and B” (and the like) as well as “one or more of A or B” (and the like) should be interpreted as covering only A, only B, or both A and B, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
processing a structured document conforming to a schema and having a plurality of rows, each row comprising a key and a value;
identifying a plurality of key/value variations associated with an instance of information;
generating a plurality of key/value variation vectors by embedding each key/value variation in a vector;
combining the plurality of key/value variation vectors into a combined vector representing the instance of information;
segmenting a transcript of speech into a segment, the segment corresponding to the instance of information;
generating a transcript segment vector by embedding the instance of information in the segment into a vector;
determining a similarity between the transcript segment vector and the combined vector;
extracting the instance of information from the transcript segment vector associated with a key from the combined vector by processing a prompt including at least a portion of the transcript segment vector and the combined vector with a generative artificial intelligence (AI) model using retrieval augmented generation (RAG); and
populating a value of a row corresponding to the key from the combined vector with the instance of information.

2. The computer-implemented method of claim 1, wherein the structured document includes a healthcare flowsheet.

3. The computer-implemented method of claim 1, wherein generating the plurality of key/value variation vectors includes generating a weight for each of the key/value variation vectors.

4. The computer-implemented method of claim 1, further comprising:
generating a plurality of examples corresponding to a plurality of instances of information.

5. The computer-implemented method of claim 4, further comprising:
generating a plurality of example vectors by embedding each of the plurality of examples into an example vector.

6. The computer-implemented method of claim 4, wherein combining the plurality of key/value variation vectors into the combined vector includes combining one or more of the plurality of example vectors into the combined vector.

7. The computer-implemented method of claim 5, wherein each example includes a text input corresponding to an instance of information.

8. The computer-implemented method of claim 1, further comprising:
storing the plurality of key/value variation vectors in a schema database.

9. The computer-implemented method of claim 5, further comprising:
storing the plurality of example vectors in an example database.

10. A computing system comprising:
a memory; and
a processor to:
process a structured document conforming to a schema and having a plurality of rows, each row comprising a key and a value;
identify a plurality of key/value variations associated with an instance of information;
generate a plurality of key/value variation vectors by embedding each key/value variation in a vector, wherein generating the plurality of key/value variation vectors includes generating a weight for each of the key/value variation vectors;
combine the plurality of key/value variation vectors into a combined vector representing the instance of information;
segment a transcript of speech into a segment, the segment corresponding to the instance of information;
generate a transcript segment vector by embedding the instance of information in the segment into a vector;
determine a similarity between the transcript segment vector and the combined vector;
extract the instance of information from the transcript segment vector associated with a key from the combined vector by processing a prompt including at least a portion of the transcript segment vector and the combined vector with a generative artificial intelligence (AI) model using retrieval augmented generation (RAG); and
populate a value of a row corresponding to the key from the combined vector with the instance of information.

11. The computing system of claim 10, wherein the structured document includes a healthcare flowsheet.

12. The computing system of claim 10, wherein the processor is further configured to:
generate a plurality of examples corresponding to a plurality of instances of information.

13. The computing system of claim 12, wherein the processor is further configured to:
generate a plurality of example vectors by embedding each of the plurality of examples into an example vector.

14. The computing system of claim 12, wherein combining the plurality of key/value variation vectors into the combined vector includes combining one or more of the plurality of example vectors into the combined vector.

15. A computer program product residing on a non-transitory computer readable medium having a plurality of instructions stored thereon which, when executed by a processor, cause the processor to perform operations comprising:
processing a structured document conforming to a schema and having a plurality of rows, each row comprising a key and a value;
identifying a plurality of key/value variations associated with an instance of information;
generating a plurality of key/value variation vectors by embedding each key/value variation in a vector;
combining the plurality of key/value variation vectors into a combined vector representing the instance of information;
segmenting a transcript of speech into a segment, the segment corresponding to the instance of information;
generating a transcript segment vector by embedding the instance of information in the segment into a vector;
determining a similarity between the transcript segment vector and the combined vector;
extracting the instance of information from the transcript segment vector associated with a key from the combined vector by processing a prompt including at least a portion of the transcript segment vector and the combined vector with a generative artificial intelligence (AI) model using retrieval augmented generation (RAG); and
populating a value of a row corresponding to the key from the combined vector with the instance of information.

16. The computer program product of claim 15, wherein the structured document includes a healthcare flowsheet.

17. The computer program product of claim 15, wherein the operations further comprise:
generating a plurality of examples corresponding to a plurality of instances of information.

18. The computer program product of claim 17, wherein the operations further comprise:
generating a plurality of example vectors by embedding each of the plurality of examples into an example vector.

19. The computer program product of claim 15, wherein combining the plurality of key/value variation vectors into the combined vector includes combining one or more of the plurality of example vectors into the combined vector.

20. The computer program product of claim 15, further comprising:
storing the plurality of key/value variation vectors in a schema database.
